(19) United States
(12) Patent Application Publication
(10) Pub. No.: US 2010/0255122 A1
(43) Pub. Date: Oct. 7, 2010

(54) EDIBLE GELATIN BOWEL PREPARATION AND BOWEL CLEANSING METHOD

(76) Inventors: Mary L. Garren, Carmel, CA (US); Lloyd R. Garren, Carmel, CA (US); Kimmie S. Garrido, Marina, CA (US); Daniel G. Luba, Monterey, CA (US)

Correspondence Address: CONNOLLY BOVE LODGE & HUTZ, LLP, P O BOX 2207, WILMINGTON, DE, 19899, US

(21) Appl. No.: 12/752,406

(22) Filed: Apr. 1, 2010

Related U.S. Application Data
(60) Provisional application No. 61/166,057, filed on Apr. 2, 2009.

(51) Int. Cl. A61K 33/14; A61P 1/10

(52) U.S. Cl. 424/679

(57) ABSTRACT

The present invention relates to an eatable gelatin bowel preparation for use in preparing an individual for a medical procedure requiring a clean colon. Specifically, an individual is provided with an edible diet comprising a laxative and a gelatin based food.

### CROSS-REFERENCE TO RELATED APPLICATION

The present application claims the benefit of provisional application Ser. No. 61/166,057, filed Apr. 2, 2009.

### BACKGROUND OF THE INVENTION

The present invention relates to a cleansing method as well as a bowel cleansing preparation, and more particularly to bowel cleansing method and an eatable gelatin bowel preparation.

Intestinal or more specifically colon related procedures are frequently done both for diagnostic and therapeutic reasons. This requires bowel cleansing prior to the procedure to maximize both benefit and safety. This cleansing process is currently done with the use of a pill or liquid laxative, and a liquid or fasting diet. Both are difficult for the patient to take and to follow due to the required volumes and palatability.

### SUMMARY OF THE INVENTION

The present invention relates to an oral nutritional dietary and laxative system and method for use in preparing an individual for an activity requiring a clean colon, such as but not limited to colonoscopy, virtual colonoscopy, barium enema, and bowel surgery.

### DETAILED DESCRIPTION OF THE INVENTION

Specifically, the present invention provides an individual with an edible, palatable diet combining a laxative, which is polyethylene glycol based, and a gelatin based food. This when taken in a specific manner will provide a sense of satiety, palatability, and good colon cleansing by facilitating stool removal. The diet minimizes stool formation and is low in both fiber and fat. It provides for a calorie base that allows an individual to do routine activities and maintain hydration. One embodiment is to provide a packaged powder that is to be mixed with warm or boiling water and refrigerated, and another embodiment is pre-made portions of solid gelatin food to be taken at specific times with a hydrating beverage.

### EXAMPLES

### Embodiment I

A. Mix

1. 420 grams polyethylene glycol 3350

2. 5.72 grams sodium bicarbonate

3. 11 2 grams sodium chloride

4. 1.48 grams potassium chloride

B. Mix

Combine above mixture A with 36 grams of unflavored gelatin and 36 grams of sugar free Jell-O® gelatin dessert.

C. Mix

Mix 12 cups of hot water and B Mix until all solids are dissolved, then slowly add 4 cups of cool water. Mix well and refrigerate for 4 hours.

D. Above makes 16 cups of C Mix this needs to be prepared the day prior to beginning the bowel cleansing or a pre-prepared formulation could be purchased.

Method of Cleansing

1. Eat low fiber high protein breakfast such as eggs, juice 6 oz, 8 oz coffee, tea or other beverage

2. Lunch Boost® or Ensure® low fiber and 10 or more oz beverage

3. Mid-afternoon snack—2.5 cups C Mix with sports drink 6-10 oz

4. Dinner 3.5 cups C Mix with sports drink 6-10 oz

5. Early evening snack 2.5 cups C Mix with 6-10 oz water

6. Bedtime snack 2.5 cups C Mix with 6-10 oz water

7. In am 6 hours before procedure, 3.5 cups C Mix with 6-10 oz water

8. 4 hours prior to procedure 1.5 cups C Mix with 8-12 oz of water or other clear liquid

### Embodiment 2

A. Mix

1. 100 grams polyethylene glycol 3350

2. Sodium sulfate 7.5 grams

3. Sodium chloride 2.69 grams

4. Sodium ascorbate 5.9 grams

5. Ascorbic acid 4.7 grams

6. Potassium chloride 1.02 grams

B. Mix

1. Combine A Mix with 20.25 grams of unflavored gelatin and 20.25 grams of sugar free Jell-O® gelatin dessert.

C. Mix

1. Mix 7 cups of hot water with B Mix—mix until all solids are dissolved then add 2 cups of cold water-mix well and refrigerate for at least 4 hours.

D. 9 cups of C Mix are formed. This needs to be prepared the day prior to beginning the bowel cleansing or it could be purchased pre-prepared

Method of Cleansing

1. Eat low fiber high protein breakfast such as 3 eggs, 18-20 or more oz beverage (juice, coffee, tea, etc.)

2. Lite lunch—either Boost® or Ensure® 1 can and 8-10 oz fluid

3. Mid-afternoon snack 2 cups C Mix and 8 oz sports drink

4. Dinner 3 cups C Mix 8-10 oz sports drink

5. Evening snack 2 cups C Mix with 8-10 oz water

6. In am 6 hours before procedure 2 cups C Mix with 8-10 oz water

Halflytely contains: polyethylene glycol 3350 210 G, sodium bicarbonate 2.86 G, sodium chlorides 5.6 G, potassium chloride 0.74 G.

When mixed with water to make 2 liters, this solution contains PEG-3350 31.3 mmol/l, sodium 65 mmol/l, chloride 53 mmol/l, bicarbonate 17 mmol/l and potassium 5 mmol/l.

For halflytely prep I measure out 24 tablespoons of (PEG 3350 PREP) 100 gram.

Separately, one package of 0.6 ounces & 1 tablespoon of sugar free jell-o & three envelopes (5.4 grams) of knox gelatin (used to thicken the gelatin) will be placed in a dry mixer and mixed together, blending well for three minutes. Set separately aside all dry mix on a 15×15 sized piece of waxed paper.

In a four cup measuring container, pour 1 cup of bottled drinking water and heat to 97 degrees Fahrenheit. When the water is brought to the appropriate temperature, six tablespoons of halflytely powder are added and whisked for five minutes until ingredients are solubilized.

In yet another four cup container, add one cup of bottled drinking water and heat using microwave heat for one minute and thirty seconds. When the water is appropriately heated, slowly add the dry jell-o and knox gelatin mixture, while simultaneously whishing the water and dry ingratiates together until all particles are dissolved. The placement of food gauge thermometer into the mixture is then made, delineating the temperature from the microwave oven between approximately 130-135 degrees Fahrenheit. Slowly add chilled bottled water until the volume of the solution reaches the two-cup mark on measured container. The gelatin slurry should then be cooled to reach a temperature of 97 degrees fahrenheit.

When the gelatin mix has cooled to 97.0 degrees fahrenheit, add the one cup of liquid halflytely with the jell-o and knox gelatin slurry constantly stirring the solution, adding more chilled water until the solution reaches the four-cup mark on the measuring container. This entire process yields four cups of edible gelatin bowel cleansing preparation. The total colon cleansing preparation will require the mixing of an amount equal to 16 cups of gelatinized product.

Chill the halflytely, jell-o and gelatin matrix in the refrigerator for two hours.

Entire product consumed all the halflytely prep/21.6 ounces & 4 tablespoons sugar free jell-o/& 21.6 grams (3 envelopes of knox gelatin) 16 cups of bottled drinking water chilled and heated to appropriate temperatures.

Prior art of interest includes Re 36,288, U.S. Pat. No. 5,985,339 and U.S. Pat. No. 6,866,873, and these US patent disclosures are incorporated herein in their entirety for all useful purposes.

1. A method of preparing an individual's colon for a colon procedure without fasting or taking large volumes of liquid or pill laxatives, the method comprising the following steps:
administering to the individual a dietary regimen and a gelatin based food in which is combined a polyethylene based laxative which is to be consumed in the 24 hour period prior and up to several hours before the procedure.

2. The method of claim 1, wherein the preparation consists of a light breakfast of choice high in protein low in fat and fiber and a sports beverage 8-16 oz followed by a late lunch consisting of 1-3 cups of the gelatin based laxative and 8-16 oz of sports drink and dinner the same as lunch and either a similarly composed early evening snack or in the morning several hours before the procedure to thereby complete the colon cleansing.

3. An eatable gelatin bound preparation comprising:
a gelatin portion selected from the group consisting of gelatin, adipic acid, disodium phosphate, maltodextrin, fumaric acid, sugar or artificial sweeteners, food dye, bha, and sodium citrate; and
laxative portion selected from the group consisting of PEG-3350, sodium chloride, potassium chloride, sodium bicarbonate, sodium sulfate, sodium ascorbate and ascorbic acid.

4. A method as in claim 1 wherein the gelatin based laxative combination includes:
27.8 grams of the gelatin portion mixed with the laxative portion comprising 121.8 grams-438.94 grams.
